(19) United States
(12) Patent Application Publication
(10) Pub. No.: US 2016/0123986 A1
(43) Pub. Date: May 5, 2016

(54) METHOD FOR DIAGNOSING CANCER USING SECRETED RIBOSOMAL PROTEIN S3

(71) Applicant: KOREA UNIVERSITY RESEARCH AND BUSINESS FOUNDATION, Seoul (KR)

(72) Inventor: Joon Kim, Seoul (KR)

(73) Assignee: KOREA UNIVERSITY RESEARCH AND BUSINESS FOUNDATION, Seoul (KR)

(21) Appl. No.: 14/590,968

(22) Filed: Jan. 6, 2015

(30) Foreign Application Priority Data
Oct. 29, 2014 (KR) 10-2014-0148486

(51) Int. Cl. G01N 33/574

(52) U.S. Cl. CPC G01N 33/57496; G01N 2333/4703

(57) ABSTRACT

A method for diagnosing cancer using secreted ribosomal protein S3 (rpS3) is described, and more particularly, a method for diagnosing cancer or cancer metastasis using ribosomal protein S3 (rpS3) that is expressed in and secreted from cancer cells. Measuring the amount of extracellularly secreted rpS3 protein according to the present disclosure is highly useful for diagnosing cancer or cancer metastasis in a rapid and convenient manner using a biological sample such as blood.

### CROSS-REFERENCE TO RELATED APPLICATION

This application claims priority under the provisions of 35 USC 119 to Korean Patent Application No. 10-2014-0148486 filed Oct. 29, 2014. The disclosure of Korean Patent Application No. 10-2014-0148486 is hereby incorporated herein by reference, in its entirety, for all purposes.

### TECHNICAL FIELD

The present invention relates to a method for diagnosing cancer using secreted ribosomal protein S3 (rpS3), and more particularly, to a method for diagnosing cancer or cancer metastasis using ribosomal protein S3 (rpS3) that is expressed in and secreted from cancer cells.

### BACKGROUND ART

Proteins are involved in all physiological processes, from cell signaling to tissue remodeling to organ function. Thus, protein synthesis in cells is a very important process in which ribosome plays a functional role. Ribosome is a cell organelle that functions to synthesize proteins by joining amino acids, and is composed of large and small subunits that are made of ribosomal proteins and ribosomal RNAs.

Ribosomal protein S3 (rpS3) that is a component of ribosome is located on the outer surface of the 40S subunit, and is cross-linked to the initiation factor eIF2 and eIF3. rpS3 has a nuclear localization signal in its N-terminal region, which suggests that rpS3 functions in the cytoplasm and that the repair function operates in the nucleus. In addition, many ribosomal proteins have secondary functions in replication, transcription, RNA processing, DNA repair, and malignant transformation.

The rpS3 gene is located at human chromosome 11q13.3-q13.5. Particularly, it was reported that the rpS3 gene is overexpressed in colorectal cancer patients. Thus, the rpS3 gene product is absent or modified in XP-D, and is overexpressed in colorectal cancer, and is highly likely to be associated with other cancers. In addition, in studies on the 11q13.3-q13.5 region, it is known that the structural abnormality and amplification of the gene frequently occur and that the gene is overexpressed upon the development of human cancers such as multiple endocrine neoplasia type, breast carcinoma, and B cell neoplasia (Pogue-Geile et al., Mol. Cell. Biol., 11: 3842-3849, 1991).

Intensive studies on the identification of the cause of cancer, methods for diagnosis of cancer, and methods for treatment of cancer have been conducted for past years, a certain therapeutic agent capable of effectively treating cancer has not yet been developed. Thus, it can be the most effective method to find cancer in an early stage and remove cancer cells or inhibit the growth of cancer cells by surgical operations or drug therapy.

Cancers usually result in symptoms such as the invasion of cancer cells into the surrounding tissues due to their growth, or lymph node metastasis. However, many cancers progress without any subjective symptoms in an early stage, and thus are found after metastasis to other organs in many cases. In order to reduce the rate of death caused by cancer, early diagnosis is required, and is generally performed by ultrasonic test, CT based on X-rays, MRI imaging, etc., and finally, tissue biopsy. However, methods such as tissue biopsy cause pain in patients, and thus, there is a need for the development of an examination method capable of diagnosing cancer in a more convenient and rapid manner.

The levels of a number of cellular proteins in the body fluids of various cancer patients appear to increase or decrease. Whether a protein showing a cancer cell-specific expression pattern is present in patients or the analysis of the expression level of the protein can be used for the diagnosis of cancer or cancer metastasis by a method of detecting a cancer-specific antigen.

Accordingly, the present inventors have made extensive efforts to develop a method capable of early diagnosing cancer in a convenient and rapid manner using a cancer-specific marker present in biological samples such as blood or body fluids, and as a result, have found that ribosomal protein S3 (rpS3) is overexpressed in and secreted from cancer cells, thereby completing the present invention.

### DISCLOSURE OF INVENTION

It is an object of the present invention to provide a method of measuring the amount of rpS3 protein, expressed in and secreted from cancer cells, in an isolated biological sample in order to diagnose cancer or cancer metastasis.

To achieve the above object, the present invention provides a method for diagnosing cancer or cancer metastasis, the method comprising the steps of: (a) measuring the amount of rpS3 protein, expressed in and secreted from cancer cells, in an isolated biological sample; and (b) comparing the measured amount of the rpS3 protein with that in a control sample.

Other features and embodiments of the present invention will be more apparent from the following detailed descriptions and the appended claims.

### BRIEF DESCRIPTION OF THE DRAWINGS

FIG. 1 shows the results of analyzing the extracellular secretion of rpS3 protein in culture media of human fibrosarcoma cells (A) and mouse plasmacytoma cells (B).

FIG. 2 shows the results of analysis conducted to confirm increases in the extracellular secretion of rpS3 protein in culture media of human bronchial epithelial cells (BEAS-2B) and malignant human bronchial epithelial cells.

FIG. 3 shows the results of comparing the extracellular secretion of rpS3 protein in culture media of mouse plasmacytoma cells and malignant plasmacytoma cells having resistance to an anticancer substance.

FIG. 4 shows the results of comparing the extracellular secretion of rpS3 protein by cancer metastasis in culture media of breast cancer cells.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods are those well known and commonly employed in the art.

In the present invention, it was found that rpS3 protein overexpressed in a cancer cell line is extracellularly secreted and that the extracellular secretion of rpS3 protein increases as the malignancy of malignant cancer cells increases and as cancer metastasis progresses.

Thus, in one aspect, the present invention is directed to a method for diagnosing cancer or cancer metastasis, the method comprising the steps of: (a) measuring the amount of rpS3 protein, expressed in and secreted from cancer cells, in an isolated biological sample; and (b) comparing the measured amount of the rpS3 protein with that in a control sample.

In the present invention, the isolated biological sample may be diagnosed as cancer or cancer metastasis if the amount of the rpS3 protein in the isolated biological sample is determined to be higher than that in the control sample in step (b). rpS3 protein is overexpressed in and secreted from cancer cells, and the extracellular secretion of rpS3 protein increases as the malignancy of malignant cancer cells increases and as cancer metastasis progresses. Thus, cancer or cancer metastasis can be diagnosed depending on the secreted level of rpS3 protein, determined by measuring the amount of extracellularly secreted rpS3 protein.

In the present invention, the cancer is selected from the group consisting of fibrosarcoma, plasmacytoma, bronchial cancer, leukemia, breast cancer, ovarian cancer, uterine cancer, skin cancer, bladder cancer, prostate cancer, renal cancer, thyroid cancer, esophageal cancer, stomach cancer, liver cancer, pancreatic cancer, lung cancer, brain tumor, osteosarcoma, malignant hemangioma, and malignant lymphoma, but is not limited thereto.

In the present invention, the sample is preferably selected from the group consisting of tissue, cell, blood, serum, plasma, saliva, cerebrospinal fluid, sweat, urine, ascetic fluid, and peritoneal fluid, but is not limited thereto. The sample can be isolated in a simple and easy manner, and thus can overcome the inconvenience of conventional tissue biopsy and enables cancer or cancer metastasis to be diagnosed in a more rapid manner. For example, the development of cancer, and furthermore, cancer metastasis, can be diagnosed by measuring the level of rpS3 protein in a regular blood test.

In the present invention, the level of rpS3 protein may be measured using an antibody or aptamer that binds specifically to the rpS3 protein.

The antibody is intended to include all “antibodies”, including polyclonal antibodies, monoclonal antibodies and recombinant antibodies. The term “antibody” means a specific protein molecule that is directed against an antigenic site. A polyclonal antibody can be produced according to a method widely known in the art by injecting the rpS3 protein antigen into an animal, collecting blood from the blood, and isolating antibody-containing serum from the blood. This polyclonal antibody can be produced from any animal species hosts such as goats, rabbits, sheep, monkeys, horses, pigs, mouse, rat, cattle, dogs, etc. A monoclonal antibody can be produced by any methods well known in the art such as a hybridoma method and a phage antibody library technique. The antibody produced by the above-described method can be isolated and purified by methods such as gel electrophoresis, dialysis, salt precipitation, ion exchange chromatography or affinity chromatography. Moreover, the antibody that is used in the present invention includes functional fragments of antibody molecules, as well as a complete form having two full-length light chains and two full-length heavy chains. The functional fragments of antibody molecules refer to fragments having at least an antigen-binding function, and include Fab, F(ab′), F(ab′)2, and Fv.

Analysis methods for measuring the amount of the protein include, but are not limited to, immunoblotting (Western blotting), ELISA, radioimmunoassay, radioimmunodiffusion, Ouchterlony immunodiffusion, rocket immunoelectrophoresis, immunohistostaining, immunoprecipitation assay, complement fixation assay, FACS, and protein chip assay.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Examination of rpS3 Secretion from Cancer Cell Lines

Human fibrosarcoma cells (HT1080), mouse plasmacytoma cells (MPC11) and human bronchial epithelial cells (BEAS-2B) were cultured in 10% FBS (Thermo Scientific, USA)-containing DMEM media (Thermo Scientific, Rockford, USA), and then cultured in serum-free DMEM media for 16 hours. Next, the media were collected and centrifuged at 1,000 rpm for 3 minutes to remove dead cells, and the precipitated pellet was removed. Then, each of the supernatants was concentrated 200-fold using an Amicon Ultra-15 Centrifugal Filter Device (Millipore, USA). Each of the concentrated supernatants was developed on 12% SDS-PAGE, and then transferred to a PVDF (polyvinylidene difluoride) membrane, which was then blocked with a blocking solution for 1 hour. Next, three ribosomal proteins (S3, L28 (Millipore, USA) and L11 (Santa Cruz Biotechnology, USA)) were analyzed by Western blotting, and the presence of protein secreted into the cell-free media in the concentrates for analysis was examined using fibrillarin (Santa Cruz Biotechnology, USA), C23 (Santa Cruz Biotechnology, USA) and Raf1 (Santa Cruz Biotechnology, USA) antibodies. As a negative (−) control, a medium in which cells were not cultured was used, and as a positive (+) control, a lysate of each of the cancer cell lines was used.

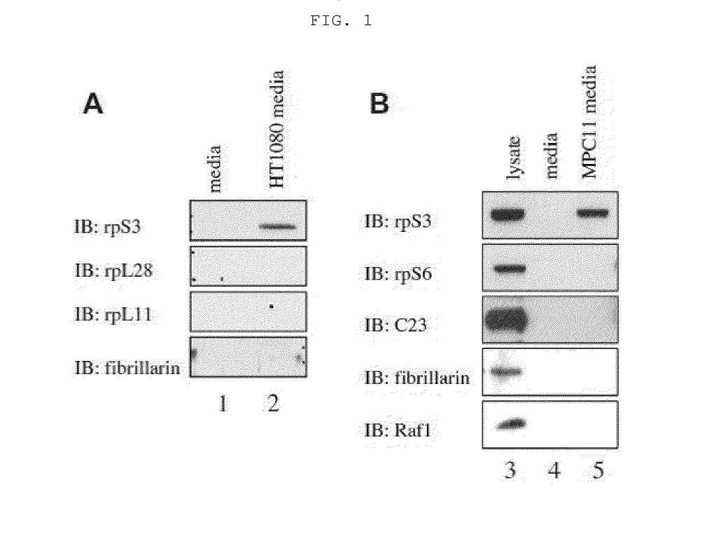

As a result, only ribosomal protein S3 appeared in the culture media of the human fibrosarcoma cells (HT1080) and the rat plasmacytoma cells (MPC11) (FIG. 1), and the secretion of ribosomal protein S3 from the human bronchial epithelial cells was also observed.

### Example 2

### Examination of Increase in rpS3 Secretion from Malignant Cancer Cell Lines

### 2-1: Malignant Human Bronchial Epithelial Cells

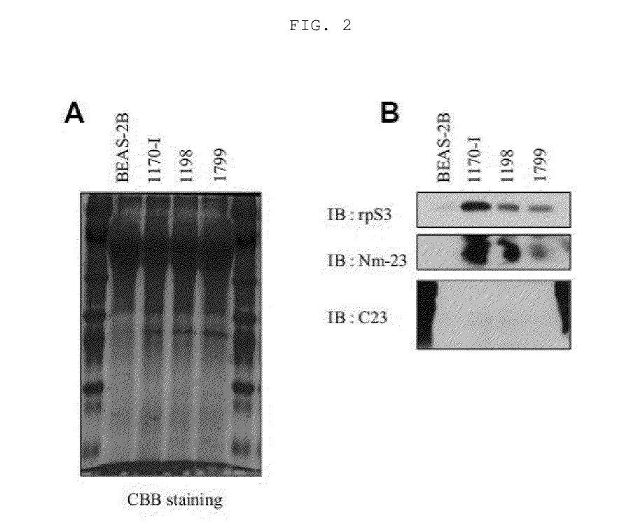

Malignant cells (1799, 1198 and 1170-1), constructed by treating human bronchial epithelial cells (BEAS-2B) with a cigarette smoke concentrate, were cultured in the same manner as described in Example 1, and then the culture media were concentrated, after which SDS-PAGE was stained with Coomassie brilliant blue to quantify the amount of protein (FIG. 2A). Western blotting for rpS3 was also performed in the same manner as described in Example 1, and the malignancy of the malignant cells was compared using known NM-23 (Santa Cruz Biotechnology) antibody. The degree of malignancy of the malignant cells appeared in the order of 1799, 1198, and 1170-1 (FIG. 2B).

The secretion of rpS3 from malignant human bronchial epithelial cells was measured by Western blotting, and as a result, it could be seen that the secretion of the rpS3 protein from the malignant cells increased as the malignancy of the malignant cells increased, like the secretion of the NM-23 protein that increased as the malignancy of the malignant cells increased (FIG. 2).

### 2-2: Malignant Mouse Plasmacytoma Cells

In order to construct cells having resistance to anticancer doxorubicin (Calbiochem, United Kingdom) from mouse plasmacytoma cells (MPC11), mouse plasmacytoma cells were cultured with doxorubicin for 45 days to construct malignant mouse plasmacytoma cells (MPC11-Dox) having resistance to anticancer doxorubicin. The malignant mouse plasmacytoma cells (MPC11-Dox) were cultured in the same manner as described in Example 1, and then the culture medium was concentrated, and the secretion of the rpS3 protein from the cells was measured.

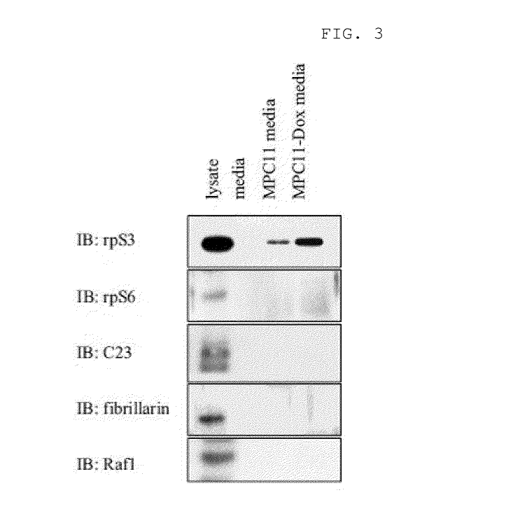

As a result, the secretion of the rpS3 protein was higher in the culture medium of the malignant mouse plasmacytoma cells (MPC11-Dox) having doxorubicin resistance than in the culture medium of the mouse plasmacytoma cells (MPC11) (FIG. 3). In other words, it could be seen that the extracellular secretion of ribosomal protein S3 (rpS3) increased as the malignancy of the malignant cells increased.

### Example 3

### Examination of the Increase in rpS3 Secretion by Metastasis of Cancer Cells

In the same manner as described in Example 1, breast cancer cell lines (MCF7 and MDA-MB-231) were cultured in 10% FBS (Thermo Scientific, USA)-containing DMEM media(Thermo Scientific, Rockford, USA), and then cultured in serum-free DMEM media for 16 hours. Next, the media were collected and centrifuged at 1,000 rpm for 3 minutes to remove dead cells, and the precipitated pellet was removed. The supernatant was not concentrated using Amicon Ultra-15, and the secretion of ribosomal protein S3 in the media was examined. The collected media were subjected to protein quantification, and used in an amount of 151 μg/ml. As a control, DMEM medium was used. Also, ELISA (enzyme-linked immunosorbent assay) was performed using 2.5 μg/ml of ribosomal protein S3 antibody.

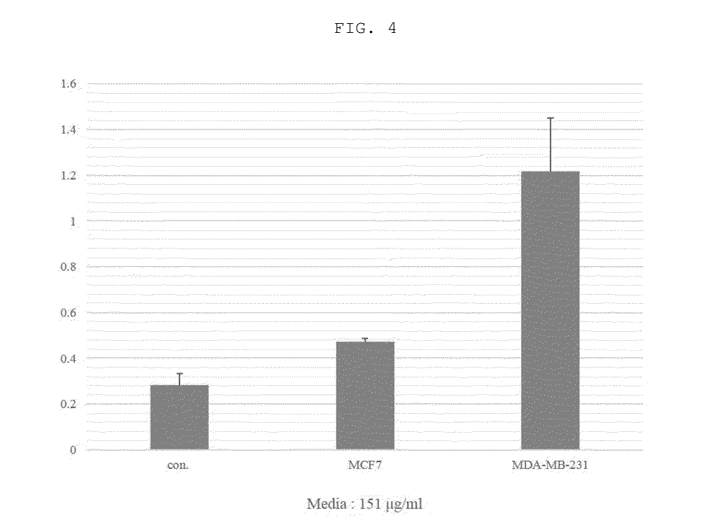

As a result, it was shown that the MDA-MB-231 cell line having higher metastasis ability compared to MCF7 secreted a significantly large amount of the rpS3 protein (FIG. 4). This suggests that the extracellular secretion of ribosomal protein S3 increases as the metastasis ability of cells increases.

### INDUSTRIAL APPLICABILITY

As described above, measuring the amount of extracellularly secreted rpS3 protein according to the present invention is highly useful for diagnosing cancer or cancer metastasis in a rapid and convenient manner using a biological sample such as blood.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for diagnosing cancer or cancer metastasis, the method comprising the steps of:
(a) measuring the amount of rpS3 protein, expressed in and secreted from cancer cells, in an isolated biological sample; and
(b) comparing the measured amount of the rpS3 protein with that in a control sample.

2. The method of claim 1, wherein the isolated biological sample is diagnosed as cancer or cancer metastasis if the amount of the rpS3 protein in the isolated biological sample is determined to be higher than that in the control sample in step (b).

3. The method of claim 1, wherein the cancer is selected from the group consisting of fibrosarcoma, plasmacytoma, bronchial cancer, leukemia, breast cancer, ovarian cancer, uterine cancer, skin cancer, bladder cancer, prostate cancer, renal cancer, thyroid cancer, esophageal cancer, stomach cancer, liver cancer, pancreatic cancer, lung cancer, brain tumor, osteosarcoma, malignant hemangioma, and malignant lymphoma.

4. The method of claim 1, wherein the sample is selected from the group consisting of tissue, cell, blood, serum, plasma, saliva, cerebrospinal fluid, sweat, urine, ascetic fluid, and peritoneal fluid.

5. The method of claim 1, wherein the amount of rpS3 protein is measured using an antibody or aptamer that binds specifically to the rpS3 protein.

6. The method of claim 1, wherein the amount of the protein is measured using any one selected from the group consisting of immunoblotting, ELISA, radioimmunoassay, radioimmunodiffusion, Ouchterlony immunodiffusion, rocket immunoelectrophoresis, immunohistostaining, immunoprecipitation assay, complement fixation assay, FACS, and protein chip assay.
